(19) United States
(12) Patent Application Publication
(10) Pub. No.: US 2014/0199752 A1
(43) Pub. Date: Jul. 17, 2014

(54) REDUCTION OF CULTURE VISCOSITY BY MANGANESE ADDITION

(71) Applicants: Jon Martin Persson, Bjaerred (SE); Niels Banke, Soeborg (DK)

(72) Inventors: Jon Martin Persson, Bjaerred (SE); Niels Banke, Soeborg (DK)

(73) Assignee: NOVOZYMES A/S, Bagsvaerd (DK)

(21) Appl. No.: 14/238,110

(22) PCT Filed: Aug. 3, 2012
(86) PCT No.: PCT/EP2012/065241

§ 371 Date: Feb. 10, 2014

Related U.S. Application Data
(60) Provisional application No. 61/524,625, filed on Aug. 17, 2011.

(30) Foreign Application Priority Data
Aug. 12, 2011 (EP) 11177428.7

(51) Int. Cl. C12N 9/28

(52) U.S. Cl. CPC C12N 9/2417
USPC 435/202; 435/222; 435/221

(57) ABSTRACT

A method of producing an enzyme of interest in a fed-batch cultivation comprising: a) cultivating a microorganism in a culture medium conducive to its growth wherein the microorganism produces the enzyme of interest; and b) adding a manganese compound to the culture medium one or more times during the cultivation.

### TECHNICAL FIELD

The present invention relates to a method of reducing broth viscosity during a fermentation wherein an enzyme of interest is produced.

### BACKGROUND ART

Bacterial and fungal microorganisms are workhorses for industrial microbiology as they are used for the commercial production of many different therapeutics (e.g. penicillin and cephalosporin), pharmaceutical proteins (e.g. insulin), enzymes (e.g. proteases and amylases), and commodity chemicals (e.g. citric acid).

A cultivation with a high viscosity of the cultivation broth has a reduced oxygen transfer compared to a cultivation with a lower viscosity under identical conditions (e.g. same pressure, temperature, aeration, and agitation). In processes where oxygen is consumed, an increased viscosity has to be compensated with an, often very costly, increase in aeration and/or agitation to keep the same oxygen tension in the cultivation medium. Alternatively, the oxygen consumption has to be reduced, often resulting in less effective processes and thereby lower yields of the desired product.

There have been many attempts to reduce the viscosity in fed-batch cultivations, e.g., WO 03/029439 discloses a method of reducing the broth viscosity by adding the carbohydrate during fermentation in a cyclic pulse dosing/pause way wherein the pulse dosing time is shorter than the pause time.

The use of a manganese compound as a trace element in culture media is known, see e.g., Electronic Journal of Biotechnology, Vol. 5, 2002, pages 110-117.

### SUMMARY OF THE INVENTION

The present inventors have found that the broth viscosity of a cultivation medium may be reduced significantly by adding a manganese compound during the cultivation, so we claim:

A method of producing an enzyme of interest in a fed-batch cultivation comprising:
a) cultivating a microorganism in a culture medium conducive to its growth wherein the microorganism produces the enzyme of interest; and
b) adding a manganese compound to the culture medium one or more times during the cultivation.

### DETAILED DISCLOSURE OF THE INVENTION

The present invention discloses a method of producing an enzyme of interest in a fed-batch cultivation wherein a manganese compound is added to the culture medium during the cultivation.

It has been found that the viscosity of the culture medium may be reduced compared to a cultivation wherein the manganese compound is not added during cultivation.

It has also been found that the yield of the compound of interest may be increased compared to a cultivation wherein the manganese compound is not added during cultivation.

### Enzyme of Interest

The enzyme in the context of the present invention may be any enzyme or combination of different enzymes obtainable by fermentation. Accordingly, when reference is made to “an enzyme”, this will in general be understood to include both a single enzyme and a combination of more than one enzyme.

It is to be understood that enzyme variants (produced, for example, by recombinant techniques) are included within the meaning of the term “enzyme”.

In a preferred embodiment, the enzyme of interest is a hydrolase (class EC 3 according to Enzyme Nomenclature; Recommendations of the Nomenclature Committee of the International Union of Biochemistry).

In a preferred embodiment the following hydrolases are preferred:

α-amylases (3.2.1.1), β-amylases (3.2.1.2), glucan 1,4-α-glucosidases (3.2.1.3), cellulases (3.2.1.4), endo-1,3(4)-β-glucanases (3.2.1.6), endo-1,4-β-xylanases (3.2.1.8), dextranases (3.2.1.11), chitinases (3.2.1.14), polygalacturonases (3.2.1.15), lysozymes (3.2.1.17), β-glucosidases (3.2.1.21), α-galactosidases (3.2.1.22), β-galactosidases (3.2.1.23), amylo-1,6-glucosidases (3.2.1.33), xylan 1,4-β-xylosidases (3.2.1.37), glucan endo-1,3-β-D-glucosidases (3.2.1.39), α-dextrin endo-1,6-α-glucosidases (3.2.1.41), sucrose α-glucosidases (3.2.1.48), glucan endo-1,3-α-glucosidases (3.2.1.59), glucan 1,4-β-glucosidases (3.2.1.74), glucan endo-1,6-β-glucosidases (3.2.1.75), arabinan endo-1,5-α-L-arabinosidases (3.2.1.99), lactases (3.2.1.108), chitosanases (3.2.1.132), glucan 1,4-alpha-maltohydrolase (3.2.1.133), xylose isomerases (5.3.1.5), and proteases (3.4).

In a particular preferred embodiment the following hydrolases are preferred:

### Amylases:

An amylase may be the desired enzyme produced according to the invention. Chemically modified or protein engineered mutants are included. There are no limitations on the origin of the amylase of the invention. Thus, the term amylase includes not only natural or wild-type amylases, but also any mutants, variants, fragments etc. thereof exhibiting amylase activity, as well as synthetic amylases, such as shuffled amylases, and consensus amylases. Such genetically engineered amylases can be prepared as is generally known in the art, e.g., by Site-directed Mutagenesis, by PCR (using a PCR fragment containing the desired mutation as one of the primers in the PCR reactions), or by Random Mutagenesis. Amylases include alpha-amylases, beta-amylases and maltogenic amylases.

An alpha-amylase may be derived from the genus Bacillus, such as, derived from a strain of B. licheniformis, B. amyloliquefaciens, B. sultilis and B. stearothermophilus. Other alpha-amylases include alpha-amylase derived from the strain Bacillus sp. NCIB 12289, NCIB 12512, NCIB 12513 or DSM 9375, all of which are described in detail in WO 95/26397, or the alpha-amylase described by Tsukamoto et al., Biochemical and Biophysical Research Communications, 151 (1988), pp. 25-31.

Other alpha-amylases include alpha-amylases derived from a filamentous fungus, preferably a strain of Aspergillus, such as, Aspergillus oryzae and Aspergillus niger.

The desired enzyme may also be a beta-amylase, such as any of plants and microorganism beta-amylases disclosed in W. M. Fogarty and C. T. Kelly, Progress in Industrial Microbiology, vol. 15, pp. 112-115, 1979.

The desired enzyme may also be a maltogenic amylase. A “maltogenic amylase” (glucan 1,4-alpha-maltohydrolase, E.C. 3.2.1.133) is able to hydrolyze amylose and amylopectin to maltose in the alpha-configuration. A maltogenic amylase of interest is the one derived from Bacillus stearothermophilus strain NCIB 11837. Maltogenic alpha-amylases are described in U.S. Pat. Nos. 4,598,048; 4,604,355; and 6,162,628.

Commercially available amylases are DURAMYL™, TERMAMYL™, FUNGAMYL™, NATALASE™, TERMAMYL LC™, TERMAMYL SC™, LIQUIZYME-X™, NOVAMYL™, and BAN™ (Novozymes A/S), RAPIDASE™ and PURASTAR™ (from Genencor International Inc.).

### Cellulases:

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, and Trichoderma, e.g., the fungal cellulases produced from Humicola insolens, Myceliophthora thermophila, Fusarium oxysporum and Trichoderma reesei.

### Lipases:

Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) or from H. insolens. Other useful lipases are Pseudomonas lipases, e.g., lipases from P. alcaligenes, P. pseudoalcaligenes, P. cepacia, P. stutzeri, P. fluorescens, or P. wisconsinensis. Other useful lipases are obtained from Bacillus, e.g. from B. subtilis, B. stearothermophilus or B. pumilus.

### Proteases:

Suitable proteases include those of animal, vegetable or microbial origin. Microbial origin is preferred. Chemically modified or protein engineered mutants are included. There are no limitations on the origin of the protease of the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e.g., by Site-directed Mutagenesis, by PCR (using a PCR fragment containing the desired mutation as one of the primers in the PCR reactions), or by Random Mutagenesis.

In a preferred embodiment the protease is an acid protease, a serine protease or a metallo protease.

In a preferred embodiment the protease is a subtilisin. A subtilisin is a serine protease that uses a catalytic triad composed of Asp32, His64 and Ser221 (subtilisin BPN′ numbering). It includes any enzyme belonging to the NC-IUBMB enzyme classification: EC 3.4.21.62.

In a preferred embodiment the subtilisin is selected from the group consisting of subtilisin Carlsberg, subtilisin BPN′, subtilisin 147, subtilisin 309 and subtilisin 1168.

Preferred commercially available subtilisins include ALCALASE™, SAVINASE™, ESPERASE™, PRIMASE™, DURALASE™, RELASE™ EVERLASE™, OVOZYME™, CORONASE™, POLARZYME™, and KANNASE™ (Novozymes NS); MAXATASE™, MAXACAL™, MAXAPEM™, PROPERASE™, PURAFECT™, PURAFECT OXP™, FN2™, FN3™, and FN4™ (Genencor International Inc.).; and BLAP X™ (Henkel).

### Amyloglucosidases:

Suitable amyloglucosidases include those of fungal origin, especially those from filamentous fungi or yeasts, e.g., Talaromyces emersonii, Aspergillus niger and Aspergillus awamori. Chemically modified or protein engineered mutants are included.

Other preferred hydrolases are carbohydrolases, transferases, lyases, isomerases, and ligases.

### Microorganisms

The microorganism expressing the enzyme of interest according to the invention may be any microorganism that can be cultivated in a fermentor.

The microorganism according to the invention may be a bacterial strain, e.g., a Gram-positive strain such as a Bacillus, Clostridium, Enterococcus, Geobacillus, Lactobacillus, Lactococcus, Oceanobacillus, Staphylococcus, Streptococcus, or Streptomyces strain, or a Gram-negative strain such as a Campylobacter, E. coli, Flavobacterium, Fusobacterium, Helicobacter, Ilyobacter, Neisseria, Pseudomonas, Salmonella, or Ureaplasma strain.

In one aspect, the strain is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis strain, in particular a Bacillus licheniformis or a Bacillus subtilis.

In another aspect, the strain is a Streptococcus equisimilis, Streptococcus pyogenes, Streptococcus uberis, or Streptococcus equi subsp. Zooepidemicus strain.

In another aspect, the strain is a Streptomyces achromogenes, Streptomyces avermitilis, Streptomyces coelicolor, Streptomyces griseus, or Streptomyces lividans strain.

The microorganism may be a fungal strain. For example, the strain may be a yeast strain such as a Candida, Hansenula, Kluyveromyces, Pichia, Saccharomyces, Schizosaccharomyces, or Yarrowia strain; or a filamentous fungal strain such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria strain.

In another aspect, the strain is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis strain.

In another aspect, the strain is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride strain.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

### Fermentations

The present invention may be useful for any fed-batch fermentation in industrial scale, e.g., for any fermentation having culture media of at least 50 litres, preferably at least 100 litres, more preferably at least 500 litres, even more preferably at least 1000 litres, in particular at least 5000 litres.

The microorganism may be fermented by any method known in the art. The fermentation medium may be a complex medium comprising complex nitrogen and/or carbon sources, such as soybean meal, soy protein, soy protein hydrolysate, cotton seed meal, corn steep liquor, yeast extract, casein, casein hydrolysate, potato protein, potato protein hydrolysate, molasses, and the like. The fermentation medium may be a chemically defined media, e.g. as defined in WO 98/37179.

The fermentation may be performed using carbon limited conditions. Carbon limited conditions mean that the growth of microorganism is controlled by the addition of the carbon source.

The method of the invention may be useful if the microorganism produces an extracellular DNase in addition to the enzyme of interest. The DNase may be a native DNase, or a copy of a DNase may have been inserted into the microorganism of interest.

It is known that many DNases require a divalent cat ion to function. According to the present invention the amount of DNA at the end of cultivation may be lower compared to a cultivation wherein the manganese compound is not added during cultivation if the microorganism produces an extracellular DNase in addition to the enzyme of interest.

In a preferred embodiment the yield of the enzyme of interest is increased compared to a cultivation wherein the manganese compound is not added during cultivation; in particular the yield of the enzyme of interest is increased after 3 days of cultivation compared to a cultivation wherein the manganese compound is not added during cultivation.

In a preferred embodiment the viscosity is reduced compared to a cultivation wherein the manganese compound is not added during cultivation; in particular the viscosity is reduced after 5 hours of cultivation compared to a cultivation wherein the manganese compound is not added during cultivation; in particular the viscosity is reduced after 10 hours of cultivation compared to a cultivation wherein the manganese compound is not added during cultivation; in particular the viscosity is reduced after 15 hours of cultivation compared to a cultivation wherein the manganese compound is not added during cultivation; in particular the viscosity is reduced after 20 hours of cultivation compared to a cultivation wherein the manganese compound is not added during cultivation; in particular the viscosity is reduced after 25 hours of cultivation compared to a cultivation wherein the manganese compound is not added during cultivation.

### Manganese Compound

The manganese compound may be any manganese compound known in the art. The manganese compound may especially be selected from the group consisting of manganese sulphate, manganese carbonate, manganese acetate, and manganese chloride.

### Addition of Manganese Compound

The manganese compound may be added separately during the cultivation, or the manganese compound may be added together with a carbohydrate as a feed medium during the fermentation.

The person skilled in the art will know how to optimise the best way of adding the manganese compound during the cultivation for a particular cultivation: The manganese compound may be added one or more times. The manganese compound may be added once during cultivation; or it may be added twice during cultivation; or it may be added three times during cultivation; or it may be added four times during cultivation; or it may be added five times during cultivation; etc.

The manganese compound may also be added continuously during the fermentation.

In a preferred embodiment the manganese compound may start to be added just after the cultivation has begun; or the manganese compound may start to be added 1 hour after the cultivation has begun; or the manganese compound may start to be added 2 hours after the cultivation has begun; or the manganese compound may start to be added 3 hours after the cultivation has begun; or the manganese compound may start to be added 4 hours after the cultivation has begun; or the manganese compound may start to be added 5 hours after the cultivation has begun; or the manganese compound may start to be added 6 hours after the cultivation has begun; or the manganese compound may start to be added 7 hours after the cultivation has begun; or the manganese compound may start to be added 8 hours after the cultivation has begun; or the manganese compound may start to be added 9 hours after the cultivation has begun; or the manganese compound may start to be added 10 hours after the cultivation has begun; or the manganese compound may start to be added 11 hours after the cultivation has begun; or the manganese compound may start to be added 12 hours after the cultivation has begun; or the manganese compound may start to be added 13 hours after the cultivation has begun; or the manganese compound may start to be added 14 hours after the cultivation has begun; or the manganese compound may start to be added 15 hours after the cultivation has begun; or the manganese compound may start to be added 16 hours after the cultivation has begun; or the manganese compound may start to be added 17 hours after the cultivation has begun; or the manganese compound may start to be added 18 hours after the cultivation has begun; or the manganese compound may start to be added 19 hours after the cultivation has begun; or the manganese compound may start to be added 20 hours after the cultivation has begun; or the manganese compound may start to be added 21 hours after the cultivation has begun; or the manganese compound may start to be added 22 hours after the cultivation has begun; or the manganese compound may start to be added 23 hours after the cultivation has begun; or the manganese compound may start to be added 24 hours after the cultivation has begun; or the manganese compound may start to be added 25 hours after the cultivation has begun; or the manganese compound may start to be added 26 hours after the cultivation has begun; or the manganese compound may start to be added 27 hours after the cultivation has begun; or the manganese compound may start to be added 28 hours after the cultivation has begun; or the manganese compound may start to be added 29 hours after the cultivation has begun; or the manganese compound may start to be added 30 hours after the cultivation has begun; or the manganese compound may start to be added 31 hours after the cultivation has begun; or the manganese compound may start to be added 32 hours after the cultivation has begun; or the manganese compound may start to be added 33 hours after the cultivation has begun; or the manganese compound may start to be added 34 hours after the cultivation has begun; or the manganese compound may start to be added 35 hours after the cultivation has begun; or the manganese compound may start to be added 36 hours after the cultivation has begun; or the manganese compound may start to be added 37 hours after the cultivation has begun; or the manganese compound may start to be added 38 hours after the cultivation has begun; or the manganese compound may start to be added 39 hours after the cultivation has begun; or the manganese compound may start to be added 40 hours after the cultivation has begun; or the manganese compound may start to be added 41 hours after the cultivation has begun; or the manganese compound may start to be added 42 hours after the cultivation has begun; or the manganese compound may start to be added 43 hours after the cultivation has begun; or the manganese compound may start to be added 44 hours after the cultivation has begun; or the manganese compound may start to be added 45 hours after the cultivation has begun.

The manganese compound may typically be added in an amount of 10-100 mg/litre start culture medium/day (calculated as MnSO4, 1H2O). For more details see Example 1.

### Viscosity

Viscosity is a measure of the resistance of a fluid which is being deformed by either shear stress or tensile stress. In everyday terms, viscosity is “thickness” or “internal friction”. Thus, water is “thin”, having a lower viscosity, while honey is “thick”, having a higher viscosity. Put simply, the less viscous the fluid is, the greater its ease of movement (fluidity).

Viscosity describes a fluid's internal resistance to flow and may be thought of as a measure of fluid friction.

The SI physical unit of dynamic viscosity is the Pascal-second (Pa·s), (equivalent to N·s/m², or kg/(m·s)). If a fluid with a viscosity of one Pa·s is placed between two plates, and one plate is pushed sideways with a shear stress of one Pascal, it moves a distance equal to the thickness of the layer between the plates in one second. Water at 20° C. has a viscosity of 0.001002 Pa·s.

### Recovery of the Compound of Interest

A further aspect of the invention concerns the downstream processing of the fermentation broth. After the fermentation process is ended, the enzyme of interest may be recovered from the fermentation broth, using standard technology developed for the compound of interest. The relevant downstream processing technology to be applied depends on the nature of the compound of interest.

A process for the recovery of an enzyme of interest from a fermentation broth will typically (but is not limited to) involve some or all of the following steps:

1) pre-treatment of broth (e.g. flocculation)

2) removal of cells and other solid material from broth (primary separation)

3) filtration

4) concentration

5) stabilization and standardization.

Apart from the unit operations listed above, a number of other recovery procedures and steps may be applied, e.g., pH-adjustments, variation in temperature, crystallization, treatment of the solution comprising the compound of interest with active carbon, use of chromatography, and use of various adsorbents.

The invention is further illustrated in the following example which is not intended to be in any way limiting to the scope of the invention as claimed.

### Example 1

### Fed-Batch Fermentation of Bacillus licheniformis with Continuous Addition of Mn⁺⁺

A number of fed-batch Bacillus licheniformis fermentations producing an amylase of interest were conducted as described below.

The amylase sequence is disclosed in SEQ ID NO:1 (including signal sequence).

All media were sterilized by methods known in the art. Unless otherwise described, tap water was used. The ingredient concentrations referred to in the below recipes are before any inoculation.

### First Inoculum Medium:

SSB4 agar:
Soy peptone SE50MK (DMV) 10 g/l;
Sucrose 10 g/l;
Di-Sodiumhydrogenphosphate, 2H2O 5 g/l;
Potassium dihydrogenphosphate 2 g/l;
Citric acid 0.2 g/l;
Vitamins (Thiamin-hydrochloride 11.4 mg/l; Riboflavin 0.95 mg/l; Nicotinic amide 7.8 mg/l; Calcium D-pantothenate 9.5 mg/l; Pyridoxal-HCl 1.9 mg/l; D-biotin 0.38 mg/l; Folic acid 2.9 mg/l);
Trace metals (MnSO4, H2O 9.8 mg/l; FeSO4, 7H2O 39.3 mg/l; CuSO4, 5H2O 3.9 mg/l; ZnSO4, 7H2O 8.2 mg/l);

### Agar 25 g/l.

Use of deionized water.
pH adjusted to pH 7.3 to 7.4 with NaOH.

### Transfer Buffer:

M-9 buffer (deionized water is used):
Di-Sodium hydrogenphosphate, 2H2O 8.8 g/l;
Potassium dihydrogen phosphate 3 g/l;
Sodium Chloride 4 g/l;
Magnesium sulphate, 7H2O 0.2 g/l.

### Inoculum Shake Flask Medium (Concentration is Before Inoculation):

### PRK-50:

110 g/l soy grits;
Di-Sodiumhydrogenphosphate, 2H2O 5 g/l;
pH adjusted to 8.0 with NaOH/H3PO4 before sterilization.

### Make-Up Medium (Concentration is Before Inoculation):

Tryptone (Casein hydrolysate from Difco) 30 g/l;
Magnesium sulphate, 7H2O 4 g/l;
Di-Potassium hydrogen phosphate 7 g/l;
Di-Sodium hydrogenphosphate, 2H2O 7 g/l;
Di-Ammonium sulphate 4 g/l;
Potassium sulphate 5 g/l;
Citric acid 0.78 g/l;
Vitamins (Thiamin-hydrochlorid 34.2 mg/l; Riboflavin 2.8 mg/l; Nicotinic amide 23.3 mg/l; Calcium D-pantothenate 28.4 mg/l;
Pyridoxal-HCl 5.7 mg/l;
D-biotin 1.1 mg/l;
Folic acid 2.5 mg/l);
Trace metals (MnSO4, H2O 39.2 mg/l; FeSO4, 7H2O 157 mg/l; CuSO4, 5H2O 15.6 mg/l; ZnSO4, 7H2O 32.8 mg/l);
Antifoam (SB2121) 1.25 ml/l;
pH adjusted to 6.0 with NaOH/H3PO4 before sterilization.

### Feed Medium:

Sucrose 708 g/l; or
Sucrose 708 g/l+200 mg/l Manganese sulphate, 1H2O

### Procedure for Inoculum Steps:

First the strain was grown on SSB-4 agar slants for 1 day at 37° C.

The agar was then washed with M-9 buffer, and the optical density (OD) at 650 nm of the resulting cell suspension was measured.

The inoculum shake flask (PRK-50) was inoculated with an inoculum of OD (650 nm)×ml cell suspension=0.1.

The shake flask was incubated at 37° C. at 300 rpm for 20 hr.

The fermentation in the main fermentor (fermentation tank) was started by inoculating the main fermentor with the growing culture from the shake flask. The inoculated volume was 11% of the make-up medium (80 ml for 720 ml make-up media).

### Fermentor Equipment:

Standard lab fermentors were used equipped with a temperature control system; pH control with ammonia water and phosphoric acid; and a dissolved oxygen electrode to measure oxygen saturation through the entire fermentation.

### Fermentation Parameters:

### Temperature: 38° C.

The pH was kept between 6.8 and 7.2 using ammonia water and phosphoric acid
Control: 6.8 (ammonia water); 7.2 phosphoric acid
Aeration: 1.5 litre/min/kg broth weight

### Agitation: 1500 rpm

### Feed Strategy:

0 hr: 0.05 g/min/kg initial broth after inoculation
8 hr: 0.156 g/min/kg initial broth after inoculation
End: 0.156 g/min/kg initial broth after inoculation

### Experimental Setup:

The feed that contained Manganese sulphate was prepared by sterile filtration of 10 ml/I of a stock solution containing 20 g/l Manganese sulphate H2O into the standard feed containing 708 g/l sucrose. The dilution of the feed with 1% by this addition was not compensated for. The cultivation was run for three days with constant agitation. The broth viscosity was measured off-line after 1 day of cultivation.

### Addition of Mn:

The amount of Mn added during the first 20 hr. before the first sample was taken was 6 mg Mn. The average addition was 11.5 mg per litre start volume/day. All these numbers are calculated as mg Mn. The amount of MnSO4, 1H20 was 35.5 mg/litre start volume/day.

### Results:

Table 1 shows the yield given as relative activity [%] compared to the activity found for the reference cultivation with no addition of Mn (at day 3):

| Amylase | Addition of | No addition |
|---|---|---|
| activity | Mn | of Mn |
| Day 1 | 17 | 16 |
| Day 2 | 74 | 88 |
| Day 3 | 201 | 100 |

Table 2 shows the measured viscosity in an off-line sample after 20 h of cultivation for the fermentation wherein Mn was added in the feed medium:

| shear | shear | | | | normal |
|---|---|---|---|---|---|
| stress | rate | viscosity | time | Temp. | stress |
| Pa | 1/s | Pa · s | s | ° C. | Pa |
| 1.81E−03 | 2.32E−03 | 0.779 | 60.813 | 38 | 0.2135 |
| 2.87E−03 | 3.13E−03 | 0.916 | 125.81 | 38 | 0.212 |
| 4.54E−03 | 6.44E−03 | 0.7055 | 190.83 | 38 | 0.2127 |
| 7.19E−03 | 0.01866 | 0.3855 | 235.78 | 38 | 0.2113 |
| 0.01141 | 0.04294 | 0.2658 | 300.73 | 38 | 0.2111 |
| 0.01808 | 0.06357 | 0.2845 | 365.7 | 38 | 0.2135 |
| 0.02866 | 0.08064 | 0.3554 | 430.73 | 38 | 0.2106 |
| 0.04542 | 0.1075 | 0.4226 | 495.72 | 38 | 0.2114 |
| 0.07197 | 0.1596 | 0.4511 | 560.73 | 38 | 0.2082 |
| 0.1141 | 0.2617 | 0.4359 | 625.75 | 38 | 0.2084 |
| 0.1808 | 0.4553 | 0.397 | 690.7 | 38 | 0.2117 |
| 0.2864 | 0.8101 | 0.3535 | 755.73 | 38 | 0.2123 |
| 0.4536 | 1.783 | 0.2543 | 810.75 | 38 | 0.2102 |
| 0.7181 | 4.245 | 0.1692 | 865.78 | 38 | 0.2179 |
| 1.136 | 11.78 | 0.09641 | 930.78 | 38 | 0.2202 |
| 1.675 | 93.77 | 0.01787 | 995.8 | 38 | 0.1754 |
| 2.686 | 439.1 | 6.12E−03 | 1060.8 | 38 | 0.1629 |
| 4.246 | 797.1 | 5.33E−03 | 1115.8 | 38 | 0.1496 |
| 6.809 | 1076 | 6.33E−03 | 1170.7 | 38 | 0.1403 |
| 10.89 | 1429 | 7.62E−03 | 1215.8 | 38 | 0.1166 |

Table 3 shows the measured viscosity in an off-line sample after 20 h of cultivation for the fermentation with no addition of Mn in the feed medium:

| shear | shear | | | | normal |
|---|---|---|---|---|---|
| stress | rate | viscosity | time | Temp. | stress |
| Pa | 1/s | Pa · s | s | ° C. | Pa |
| 1.81E−03 | 3.32E−04 | 5.444 | 60.453 | 38 | 0.2417 |
| 2.87E−03 | 4.77E−04 | 6.01 | 125.47 | 38 | 0.2377 |
| 4.54E−03 | 7.38E−04 | 6.159 | 190.44 | 38 | 0.2375 |
| 7.20E−03 | 1.17E−03 | 6.132 | 255.44 | 38 | 0.2364 |
| 0.01141 | 1.91E−03 | 5.971 | 320.44 | 38 | 0.236 |
| 0.01809 | 3.09E−03 | 5.855 | 385.47 | 38 | 0.2336 |
| 0.02867 | 5.06E−03 | 5.672 | 450.41 | 38 | 0.2309 |
| 0.04543 | 7.88E−03 | 5.765 | 515.42 | 38 | 0.2302 |
| 0.07201 | 0.01086 | 6.63 | 580.41 | 38 | 0.2281 |
| 0.1141 | 0.01511 | 7.554 | 645.41 | 38 | 0.2247 |
| 0.1809 | 0.03123 | 5.791 | 710.44 | 38 | 0.2196 |
| 0.2867 | 0.08537 | 3.358 | 765.52 | 38 | 0.2223 |
| 0.4543 | 0.133 | 3.415 | 830.48 | 38 | 0.2242 |
| 0.7201 | 0.1723 | 4.179 | 895.44 | 38 | 0.2237 |
| 1.141 | 0.2162 | 5.279 | 960.47 | 38 | 0.2263 |
| 1.809 | 0.3576 | 5.057 | 1025.5 | 38 | 0.2333 |
| 2.866 | 1.129 | 2.539 | 1080.5 | 38 | 0.2578 |
| 4.541 | 4.67 | 0.9724 | 1135.5 | 38 | 0.2966 |
| 7.194 | 12.18 | 0.5905 | 1180.5 | 38 | 0.2998 |
| 11.33 | 65.08 | 0.1741 | 1245.6 | 38 | 0.1997 |

### CONCLUSION

The addition of Mn to the culture during the cultivation has a very significant influence on the broth viscosity as can be seen by looking at the measured viscosity (see Table 2 and Table 3). The reduced broth viscosity for the culture with addition of Mn is very desirable as it results in higher productivity (see Table 1).

1. A method of producing an enzyme of interest in a fed-batch cultivation comprising:
a) cultivating a microorganism in a culture medium conducive to its growth wherein the microorganism produces the enzyme of interest; and
b) adding a manganese compound to the culture medium one or more times during the cultivation.

2. The method according to claim 1, wherein the enzyme of interest is an amylase or a protease.

3. The method according to claim 1, wherein the microorganism is a fungus or a bacterium.

4. The method according to claim 3, wherein the bacterium is a Bacillus strain.

5. The method according to claim 4, wherein the Bacillus strain is a Bacillus licheniformis strain or a Bacillus subtilis strain.

6. The method according to claim 1, wherein the manganese compound is selected from the group consisting of manganese sulphate, manganese carbonate, manganese acetate and manganese chloride.

7. The method according to claim 1, wherein the manganese compound is added together with a carbohydrate as a feed medium.

8. The method according to claim 1 wherein the manganese compound is added continuously during the cultivation.

9. The method according to claim 1, wherein the microorganism produces an extracellular DNase in addition to the enzyme of interest.

10. The method according to claim 1, wherein the yield of the enzyme of interest is increased compared to a cultivation wherein the manganese compound is not added during cultivation.

11. The method according to claim 1, wherein the viscosity is reduced compared to a cultivation wherein the manganese compound is not added during cultivation.

12. The method according to claim 1, wherein the amount of DNA at the end of cultivation is lower compared to a cultivation wherein the manganese compound is not added during cultivation.


SEQUENCE LISTING (1 sequence)

SEQ ID NO: 1 - protein, 547 aa, Bacillus sp.
  SIGNAL (1)..(35)
  mat_peptide (36)..(547)
MLTFHRIIRKGWMFLLAFLLTVSLFCPTGQPAKAAAPFNGTMMQYFEWYLPDDGTLWTKVANEANNLSSLGITALWLPPAYKGTSRSDVGYGAYDLYDLGEFNQKGTVRTKYGTKAQYLQAIRAAHAAGMQVYADVVFDHKGGADGTEWVDAVEVNPSDRNQVISGTYQIQAWTKFDFPGRGNTYSSFKWRWYHFDGVDWDESRKLSRIYLFEGKAWDWEVDTEFGNYDYLMYADLDMDYPEVVTELKNWGPWYVLTTNIDGFRLDAVKHIKFSFFPDWLSYVRSSTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP